(19) United States
(12) Patent Application Publication
(10) Pub. No.: US 2014/0332537 A1
(43) Pub. Date: Nov. 13, 2014

(54) FEMININE HYGIENE WASTE AND STORAGE CONTAINER

(71) Applicant: KERRY L. GOODFIELD, LAGUNA NIGUEL, CA (US)

(72) Inventor: KERRY L. GOODFIELD, LAGUNA NIGUEL, CA (US)

(21) Appl. No.: 14/447,039

(22) Filed: Jul. 30, 2014

Related U.S. Application Data
(63) Continuation of application No. 13/591,037, filed on Aug. 21, 2012, now abandoned.

(51) Int. Cl. B65F 1/14; B65D 51/28; A61F 13/84

(52) U.S. Cl. CPC B65F 1/1415; A61F 13/84; B65F 1/1623; B65D 51/28; B65F 1/1426; A61F 2013/8402
USPC 220/495.06; 220/521

(57) ABSTRACT

Provided is a waste disposal device for disposing of used feminine hygiene products and for storing unused feminine hygiene products. The device includes a lower body defining a lower interior compartment and a disposal opening in communication therewith. A door is coupled to the lower body and is moveable between open and closed positions. The door is moved from the closed toward the open position to facilitate passage of a used product into the lower interior compartment for disposal. An upper body is coupled to the lower body and defines a storage compartment for unused feminine hygiene products. The upper body is selectively moveable relative to the lower body between raised and lowered positions. At least a portion of the upper body is moved away from the lower body as the upper body moves from the lowered position toward the raised position to facilitate access to the disposal opening.

### CROSS-REFERENCE TO RELATED APPLICATIONS

The present application is a continuation patent application of U.S. Patent application Ser. No. 13/591,037 filed on Aug. 21, 2012, the entire contents of which are incorporated herein by reference.

### STATEMENT RE: FEDERALLY SPONSORED RESEARCH/DEVELOPMENT

Not Applicable

### BACKGROUND

Disposal of feminine hygiene products, such as sanitary napkins, tampons, and the like in a safe, sanitary, and discrete manner may be difficult. This issue may be particularly problematic in public restrooms which may not be equipped with proper disposal equipment. As such, some women may be tempted to resort to flushing the hygiene products down the toilet. However, disposal in the toilet may lead to sewage pipe blockages, which in turn may cause backups and overflow of plumbing components. In view of the problems associated with flushing the feminine hygiene products down the toilet, women may also resort to disposal in a standard waste bin commonly found in public or private bathrooms. Although waste bin disposal avoids some of the plumbing issues noted above, disposal of the hygiene products into standard waste bins generally presents other problems, including foul odors and public health, as the feminine hygiene products may become a source of infection and disease once it is soiled. Furthermore, disposal within the standard waste bins may create an unsightly and potentially embarrassing situation if a subsequent user enters the restroom, particularly if the waste bin does not include a lid.

In view of these deficiencies, special receptacles have been designed to alleviate the above-mentioned problems. One particular receptacle is generally rectangular in shape and includes a lid which may be lifted to dispose of the hygiene product. However, such receptacles do not provide a secure and controlled environment for the disposal of the hygiene products, as the lid may be left open once the deposit of the hygiene product has taken place. Thus, any odor and infection may emanate from the open container. Furthermore, such products tend to be relatively large and take up a considerable amount of floor space, which in turn may create limited free space in the bathroom.

In addition to the safe, sanitary, and discrete disposal problems noted above, another problem commonly associated with feminine hygiene products relates to retrieval of a new, unused sanitary product. Along these lines, once a hygiene product is disposed of, oftentimes, the user will need another unused, sanitary hygiene product to replace the recently disposed hygiene product. The user may not have an unused hygiene product readily available, which may pose a problem. Furthermore, it is also understood that the user may have a supply of unused hygiene products, but the supply may not be located in a location that is easily reachable from the user's current location. For instance, the supply may be in another bathroom, or may be in a hard-to-reach cupboard or cabinet.

Therefore, there is a need in the art for an easy-to-use feminine hygiene waste and storage container which allows for sanitary disposal of feminine hygiene products, which also mitigates the emission of odor from the disposed hygiene products. Various aspects of the present invention address these particular needs, as will be discussed in more detail below.

### BRIEF SUMMARY

According to one embodiment, there is provided a waste disposal device for disposing of used feminine hygiene products and for storing unused feminine hygiene products. The waste disposal device includes a disposal compartment for storing soiled feminine hygiene products, wherein the waste disposal device is configured to seal off the disposal compartment to prevent odor, disease and the like from escaping out of the disposal compartment. Thus, when a user disposes the hygiene product within the disposal compartment, the user can be assured that subsequent users of the bathroom will not notice any odor from the used hygiene product. Furthermore, the waste disposal device includes a storage bin for storing unused hygiene products. Therefore, the user may dispose of a used hygiene product and retrieve a new hygiene product at the waste disposal device.

One particular implementation of the waste disposal device includes a lower body including a tubular lower wall, a lower end wall, and an upper end wall collectively defining a lower disposal interior compartment. The upper end wall includes a disposal opening formed therein such that the disposal opening is in communication with the lower interior disposal compartment. A door is coupled to the lower body and is moveable between open and closed positions. The door is moved from the closed position toward the open position to facilitate passage of a used feminine hygiene product through the disposal opening into the lower interior compartment for disposal of the used feminine hygiene product. The device further includes an upper body having a primary wall and a transverse wall connected to the upper primary wall to define a storage compartment for unused feminine hygiene products. The upper body is selectively moveable relative to the lower body between a raised position and a lowered position, wherein at least a portion of the upper body is moved away from the lower body as the upper body moves from the lowered position toward the raised position to facilitate access to the disposal opening.

The door and the lower body may be configured to form a fluid tight seal therebetween when the door is in the closed position. The upper body and lower body may also be configured to form a fluid tight seal therebetween when the upper body is in the lowered position. The door and the upper body may both be biased toward the closed position.

The waste disposal may further include a foot pedal coupled to the upper body. The foot pedal may be configured to selectively move the upper body between the raised and lowered positions. The foot pedal may be pivotal relative to the lower body between a rest position and an actuated position, wherein the upper body moves from the lowered position toward the raised position as the foot pedal moves from the rest position toward the actuated position.

The waste disposal device may additionally include a lid coupled to the upper body. The lid may be pivotable relative to the upper body between open and closed positions, with the lid substantially covering the storage compartment when the lid is in the closed position.

The lower wall of the device may be configured to include a door portion and a stationary portion, wherein the door portion is pivotable relative to the stationary portion to access the lower interior compartment for removal of a trash bag containing used feminine hygiene products.

A restraining member may be engaged with the upper body and configured to be engageable with unused feminine hygiene products stored in the storage compartment to mitigate movement of the products relative to the upper body as the upper body moves between the raised and lowered positions.

The present invention is best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a side sectional view of one embodiment of a feminine hygiene waste and storage container in a closed position;

FIG. 2 is a side sectional view of the feminine hygiene waste and storage container depicted in FIG. 1, wherein an upper body of the container has been pivoted to an open position relative to a lower body of the container; and

FIG. 3 is a side sectional view of the feminine hygiene waste and storage container depicted in FIGS. 1-2, wherein a lid has been lifted from the upper body to access a storage compartment within the upper body.

### DETAILED DESCRIPTION

The detailed description set forth below in connection with the appended drawings is intended as a description of certain embodiments of the present disclosure, and is not intended to represent the only forms that may be developed or utilized. The description sets forth the various functions in connection with the illustrated embodiments, but it is to be understood, however, that the same or equivalent functions may be accomplished by different embodiments that are also intended to be encompassed within the scope of the present disclosure. It is further understood that the use of relational terms such as first, second, and the like are used solely to distinguish one entity from another without necessarily requiring or implying any actual such relationship or order between such entities.

Referring now to the Figures, wherein the showings are for purposes of illustrating a preferred embodiment of the present invention only, and not for purposes of limiting the same, there is provided a waste disposal device 10 for feminine hygiene products 12. The waste disposal device 10 includes a sealed disposal compartment to trap any odor or possible contaminant which may be associated with the used feminine hygiene product 12a. Therefore, when a user disposes of a used hygiene product 12a into the device 10, the user can be assured that any odor will be trapped within the device 10 to avoid any potential unsightly or potentially embarrassing situation if a subsequent user enters the bathroom. Furthermore, a portion of the waste disposal device 10 includes a storage compartment for storing unused feminine hygiene products 12b to allow the user to quickly and easily replace the hygiene product 12a that was disposed of within the device 10. Thus, the waste disposal device 10 is uniquely configured to facilitate storage of used feminine hygiene products 12a in a safe, sanitary, and discrete manner, while at the same time provide for storage of unused feminine hygiene products 12b.

As used herein, the term “feminine hygiene products” may include, but is not limited to tampons, sanitary napkins, maxi pad, or other absorbent items commonly used for hygienic purposes. Furthermore, although the exemplary embodiment described herein is specifically tailored to feminine hygiene products, it is understood that other embodiments of the present invention may be adapted for use with diapers, bandages, or other hygienic or medical dressings known by those skilled in the art.

According to one embodiment, the waste disposal device 10 includes a relatively stationary lower body 14, and a generally pivotable upper body 16. The lower body 14 defines the storage area for disposing of the used feminine hygiene products 12a, while the upper body 16 defines a storage area for storing the unused feminine hygiene products 12b.

The lower body 14 defines an upper end portion 18 and a lower end portion 20, and includes a tubular lower wall 22 extending between the upper and lower end portions 18, 20. In the exemplary embodiment, the tubular lower wall 22 defines a generally cylindrical shape, although other shapes may be defined without departing from the spirit and scope of the present invention. The lower body 14 further includes an upper end wall 24 and a lower end wall 26 extending in spaced relation to each other and generally transverse to the longitudinal direction of the tubular lower wall 22. The upper end wall 24 is disposed adjacent the upper end portion 18 of the lower body 14, while the lower end wall 26 is disposed adjacent the lower end portion 20 of the lower body 14. The lower wall 22, upper end wall 24 and lower end wall 26 collectively define an interior disposal compartment 28 for storing the used hygiene products 12b, as will be explained in more detail below.

The upper end wall 24 includes a disposal opening 30 formed therein and in communication with the disposal compartment 28. The disposal opening 30 is configured to facilitate passage of used hygiene products 12b into the disposal compartment 28.

In the exemplary embodiment, a disposal door 32 is pivotally connected to the lower body 14 adjacent the disposal opening 30. The disposal door 32 is operative to keep the disposal opening 30 generally closed. The disposal door 32 may be selectively opened during disposal of a used hygiene product 12b (i.e., wherein a used hygiene product 12b is passed through the disposal opening 30 and into the disposal compartment 28). Once the disposal of the used hygiene product 12b is complete, the disposal door 32 is closed to seal off the disposal compartment 28 to inhibit odor or possible contaminants from escaping the disposal compartment 28.

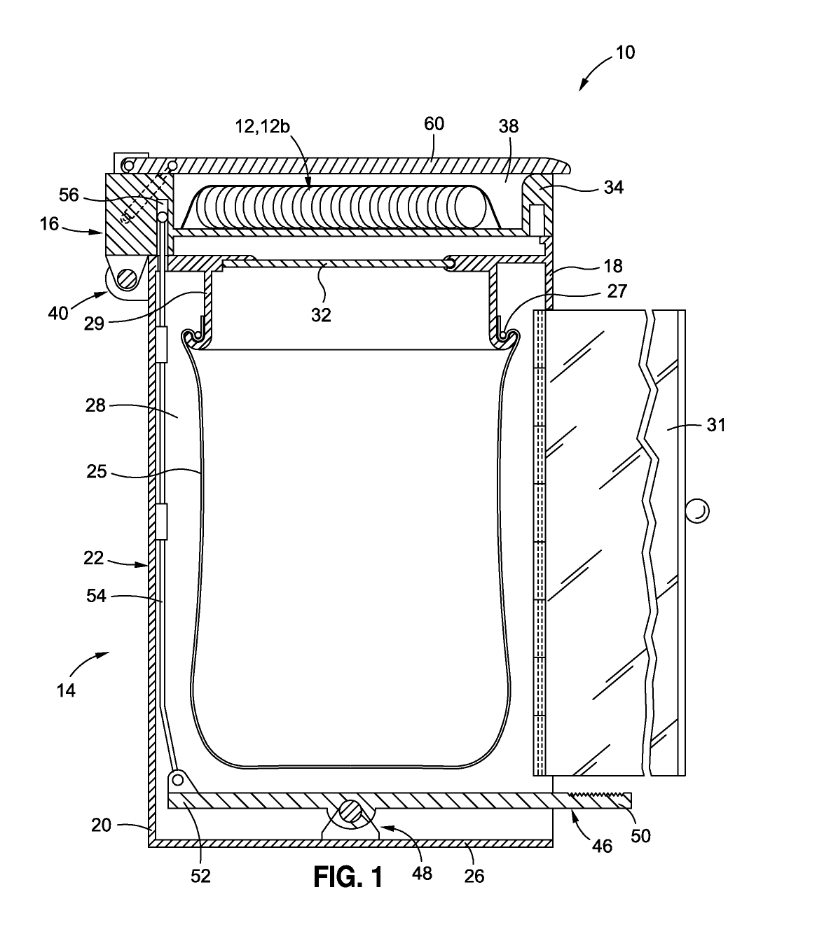

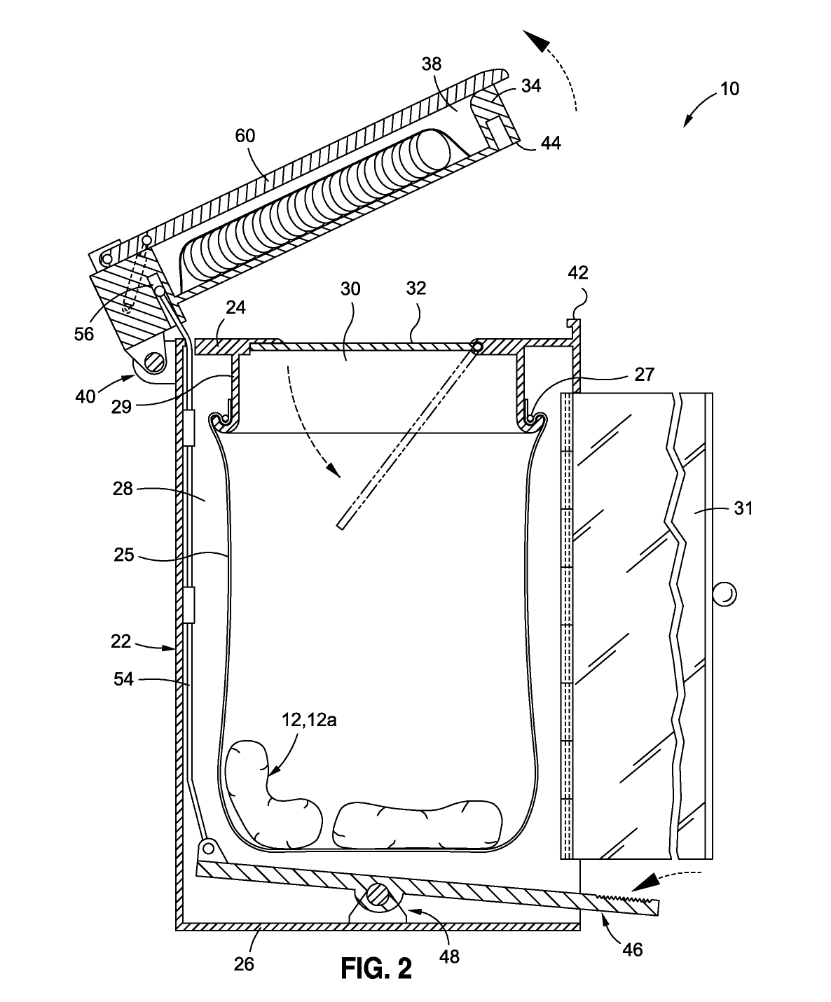

According to one embodiment, the disposal door 32 includes a generally planar member having a pivoting end portion and a free end portion. The pivoting end portion is coupled to the upper end wall 24 via a hinge which allows the disposal door 32 to pivot between a closed position and an open position. FIG. 1 shows the disposal door 32 in the closed position and FIG. 2 shows the disposal door 32 in the open position.

In the closed position, the disposal door 32 is disposed in generally co-planar alignment with the upper end wall 24 and extends across the disposal opening 30. That is to say that the plane defined by the disposal door 32 is generally parallel to the plane defined by the upper end wall 24. Furthermore, in the closed position, the free end portion of the disposal door 32 is seated adjacent the upper end wall 24. In a preferred embodiment, the disposal door 32 forms a fluid-tight seal with the upper end wall 24 when the disposal door 32 is in the closed position. In this regard, the disposal door 32 and/or the upper end wall 24 may include a rubber gasket, or the like, configured to effectuate formation of the fluid-tight seal when the disposal door 32 is in the closed position.

The disposal door 32 may be selectively moved from the closed position to the open position to open the disposal opening 30. As the disposal door 32 moves form the closed position toward the open position, the free end portion of the disposal door 32 moves from its seated position and away from the upper end wall 24 to uncover the disposal opening 30 (See FIG. 2). Thus, when the disposal door 32 is in the open position, a user may easily dispose of a used hygiene product 12b into the disposal compartment 28 through the disposal opening 30.

According to one embodiment, the disposal door 32 may be biased toward the closed position to keep the disposal opening 30 closed during periods of non-use. A spring may be used to bias the door 32 toward the closed position. Thus, in order to open the door 32, a force would need to be applied to the door 32 to overcome the biasing force. In this regard, the disposal compartment 28 preferably remains sealed when the disposal door 32 is closed to inhibit the spread of odor therefrom. It is also contemplated that a scented insert or bag may be used in the disposal compartment 28 to further mitigate the spread of undesirable odor or contaminants from the used hygiene products 12b.

Those skilled in the art will readily appreciate that the shape and configuration of the disposal door 32 is not limited to the particular embodiment depicted in the Figures. For instance, the exemplary embodiment shows the disposal door 32 and upper end wall 24 configured such that the free end portion of the disposal door 32 is lowered into the disposal compartment 28 as the door 32 moves from the closed position toward the open position. However, it is contemplated that other implementations of the waste disposal device 10 may be configured such that the free end portion of the disposal door 32 is raised or lifted away from the upper end wall 24 as the disposal door 32 moves from the closed position to the open position. In that case, the force of gravity may bias the disposal door 32 toward the closed position. It is additionally contemplated that the configuration of the disposal door 32 is not limited to a single member. Rather, other implementations of the disposal door 32 may be comprised of two or more complimentary door flaps which engage with each other when the door is in the closed position, and move away from each other when the disposal door 32 is in the open position. It is also understood that the movement of the disposal door 32 relative to the upper end wall 24 is not limited to pivotal movement. For instance, other embodiments may include a disposal door 32 which translates (i.e., slides) relative to the upper end wall 24 between the open and closed positions.

The disposal door 32 provides access to the disposal compartment 28, wherein a collection bag 25 may be placed to collect the disposed products 12a. The collection bag 25 may be retained within the disposal compartment 28 by a retaining ring 27, which rests within a retaining channel formed by a retaining wall 29. In particular, the open end portion of the collection bag 25 is clamped between the retaining ring 27 and the retaining wall 29, such that the bag opening is positioned to catch products 12a as they pass through the disposal opening 30. The collection bag 25 may be removed from the disposal compartment 28 via a door 31 formed within the lower wall 22. In this regard, the door 31 may open/pivot relative to a stationary portion of the lower wall 22.

According to one embodiment, the waste disposal device 10 is configured to cover the disposal door 32 during periods of nonuse to provide another odor/contaminant guard to substantially contain the odors/contaminants from the used hygiene products 12b within the waste disposal device 10. Referring now specifically to FIG. 1, the disposal door 32 is covered by the upper body 16, which is pivotally coupled to the lower body 16 and is in the closed/lowered position. The upper body 16 includes a pivoting end portion and a free end portion, wherein the pivoting end portion is pivotally coupled to the lower body 16. The upper body 16 includes an upper body primary wall 34 and an upper body transverse wall 36 extending in a direction that is generally perpendicular to an axis about which the primary wall 34 is disposed. The upper body primary wall 34 and upper body transverse wall 36 collectively define a storage compartment 38 for unused feminine hygiene products 12a.

The upper body 16 is selectively moveable relative to the lower body 14 between a lowered position (see FIG. 1) and a raised position (see FIG. 2). As the upper body 14 moves from the lowered position toward the raised position, at least a portion of the upper body 16 moves away from the lower body 14. Conversely, as the upper body 14 moves from the raised position toward the lowered position, at least a portion of the upper body 16 moves toward the lower body 14.

In the exemplary embodiment, the upper body 16 is pivotally connected to the lower body 14 by way of a hinge 40. Furthermore, the upper body 16 and lower body 14 are configured to physically engage with each other when the upper body 16 is in the lowered position. In particular, the lower body includes a seat 42 upon which a contact surface 44 of the upper body 16 resides when the upper body 16 is in the raised position. According to one embodiment, the upper body 16 and lower body 14 form a fluid tight seal when the upper body 16 is in the lowered position so as to mitigate the spread of odor from the waste disposal device 10.

In one implementation, the waste disposal device 10 includes a foot pedal 46 operatively coupled to the upper body 16 to control movement of the upper body 16 between the raised and lowered positions. The foot pedal 46 may be actuated by a user's foot to effectuate raising or lowering of the upper body 16. In particular, the foot pedal 46 may pivot relative to the lower body 16 between a first/rest position (see FIG. 1) and a second/actuated position (see FIG. 2) to raise and lower the upper body 16. The foot pedal 46 is connected to the lower body 16 via a foot pedal hinge 48 to allow for pivotal movement between the first and second positions. The foot pedal 46 defines a first end portion 50 and an opposing second end portion 52. The first end portion 50 extends through an opening in the lower wall 22 to allow a user to step or press on the first end portion 50 to pivot the foot pedal 46. In the exemplary embodiment, the first end portion 50 defines a roughened surface to mitigate slipping of the user's foot on the pedal 46. A strip of grippable material, such as rubber, may be applied to the first end portion 50 to enhance the engagement between the user's foot and the pedal 46.

The second end portion 52 of the foot pedal 46 is connected to an arm 54, which in turn is engaged with the upper body 16. In the exemplary embodiment, the arm 54 extends into a slot formed within the upper body 16 and engages with the upper body 16 so as to control movement of the upper body 16 between the lowered and raised positions.

As noted above, the upper body 16 preferably includes a storage compartment 38 for storage of unused feminine hygiene products 12b. The storage compartment 38 is defined by the upper body wall 34 and the upper body transverse wall 36. The bottom of the storage compartment 38 may be lined with an adhesive or gripping material to keep the feminine hygiene products 12b from slipping when the upper body 16 is moved to the raised position. It is also contemplated that a restraining strap 58 or band may be used to keep the feminine hygiene products 12b in place.

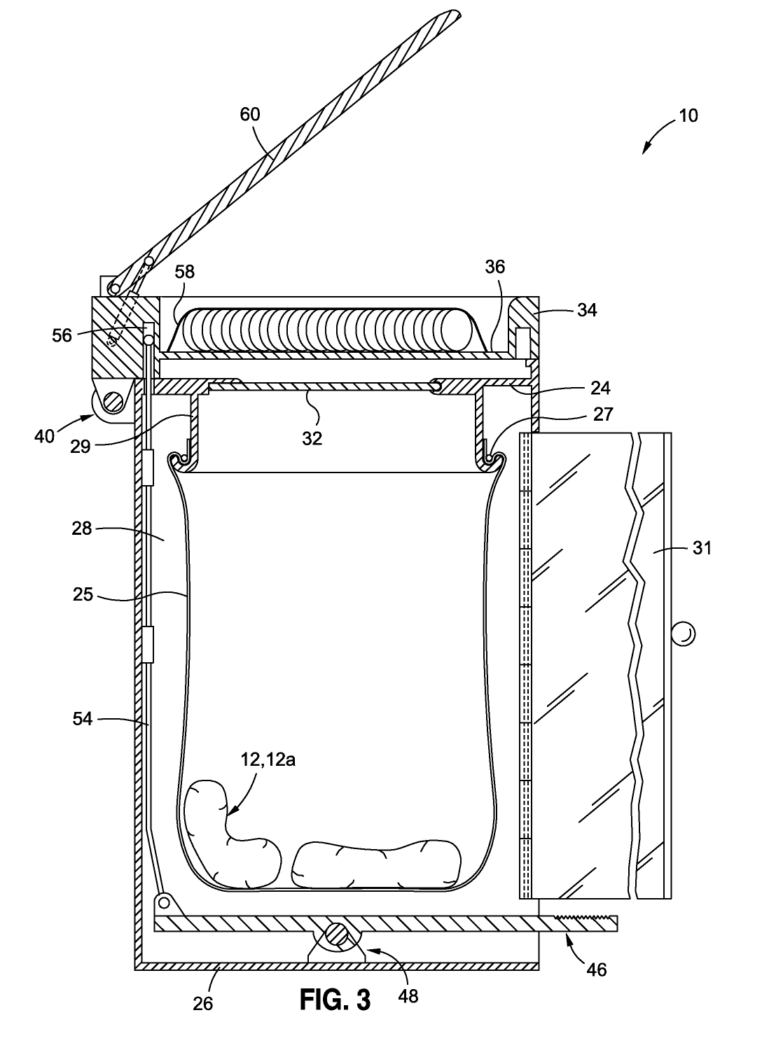

A lid 60 may be coupled to the upper body 16 to enclose the storage compartment 38. The lid 60 may be pivotable relative to the upper body 16 between an open position (See FIG. 3) and a closed position (See FIGS. 1 and 2). In the closed position, the storage compartment 38 is generally closed and a distal end of the lid 60 is disposed adjacent the upper body wall 34. The lid 60 is moved from the closed position to the open position to access the products 12b stored within the compartment 38. The lid 60 may be biased toward the closed position to keep the products 12b secured within the compartment 38.

With the basic structural features of the waste disposal device 10 being described above, the following is a description related to the operation of an exemplary embodiment of the device 10. When a user needs to dispose of a feminine hygiene product 12, the user actuates the food pedal 46 by stepping on the exposed, first end portion 50 with the user's foot to cause the foot pedal 46 to move from the first position to the second position. As the foot pedal 46 pivots, the arm 54 is raised, which in turn, raises the upper body 16 from the lowered position to the raised position. The user maintains pressure on the foot pedal 46 to keep the upper body 16 in the raised position during disposal of the feminine hygiene product 12.

With the upper body 16 raised, the disposal door 32 is exposed and accessible to the user, which allows the user to move the disposal door 32 to open the disposal opening 30. In the exemplary embodiment, the disposal door 32 is pushed into the disposal compartment 28, which causes the door 32 to pivot from its rest position. The feminine hygiene product 12 is advanced through the disposal opening 30 and is released by the user to allow the feminine hygiene product 12 to fall into the collection bag 25 placed within the disposal compartment 28 (see FIG. 2).

After the feminine hygiene product 12 is disposed of in the disposal compartment 28, the disposal door 32 is moved back to the closed position. In the exemplary embodiment, movement of the disposal door 32 from the open position to the closed position entails releasing the disposal door 32 to allow the biasing force to urge the disposal door 32 back to the closed position. As the disposal door 32 is moved back to the closed position, a fluid tight seal is formed between the disposal door 32 and the upper body 16 to mitigate odor and/or contaminant escape from the disposal compartment 28. In this regard, odor/contaminants from the feminine hygiene products 12a may be maintained within the disposal compartment 28, rather than allowing the odor/contaminant to escape into the surrounding environment.

The user may release pressure from the foot pedal 46, which allows the foot pedal 46 to revert back to the first/rest position. In the foot pedal 46 may be biased toward the first position, such that as pressure is released from the foot pedal 46, the foot pedal 46 automatically returns to the first position. According to one embodiment, the weight of the upper body 16 may provide the biasing force, which urges the foot pedal 46 back to the first position.

As the foot pedal 46 returns back to the first position, the arm 54 is lowered, which in turn, allows the upper body 16 to move from the raised position to the lowered position. When the upper body 16 returns to the lowered position, a fluid tight seal is preferably formed between the upper body 16 and the lower body 14 to provide an additional fluid-tight barrier to further mitigate odor escape from the waste disposal device 10.

The formation of a first fluid tight seal between the disposal door 32 and the upper body 16, and a second fluid tight seal between the upper body 16 and lower body 14 creates a double-seal which strengthens the odor barrier to prevent odor from seeping from the disposal compartment 28. Therefore, when the user places a used feminine hygiene product 12a within the device 10, the user will have a degree of assurance that any odor emanating from the product 12a will remain in the waste disposal device 10, and will not be noticed by subsequent users of the bathroom.

After the used feminine hygiene product 12a is disposed in the disposal compartment 28, the user may need a new, unused hygiene product 12b. Thus, the user may access the storage compartment 38 within the upper body 16 by lifting the lid 60. The lid 60 may have a lock which locks the lid 60 to the upper body 16 to keep the products 12b within the storage compartment 38 while the upper body 16 moves between the raised and lowered positions. For instance, if the lid 60 moves when the upper body 16 is in the raised position, the products 12b may fall out of the storage compartment 38. Thus, by locking the lid 60 to the upper body 16, the contents may remain securely within the storage compartment 38. The lock used to secure the lid 60 to the upper body 16 may include a push-actuated lock, wherein the lid 60 is pressed toward the upper body 16 to initially lock the lid 60 in place, and then pressed again toward the upper body 16 to unlock the lid 60 from the upper body 16. The press-actuated lock is simply one example of a locking mechanism which may be used to secure the lid 60 in place, and those skilled in the art will readily appreciate that other locking mechanisms may be used without departing from the spirit and scope of the present invention.

When the lid 60 is unlocked, the lid 60 may be lifted to access the storage compartment 38 and remove a new hygiene product 12b. After the hygiene product 12b is retrieved, the lid 60 may be returned to the lowered position. The lid 60 may be biased toward the closed position, such that it automatically returns to the closed position when released by the user. The biasing force may be applied by a spring, hydraulic arm or the like.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A waste disposal device for disposing of used feminine hygiene products and for storing unused feminine hygiene products, the waste disposal device comprising:
a lower body including a tubular lower wall, a lower end wall, and an upper end wall collectively defining a lower interior compartment, the upper end wall having a disposal opening formed therein, the disposal opening being in communication with the lower interior compartment;
a door coupled to the lower body and moveable between open and closed positions, the door being moved from the closed position toward the open position to facilitate passage of a used feminine hygiene product through the disposal opening into the lower interior compartment for disposal of the used feminine hygiene product; and
an upper body having a primary wall and a transverse wall connected to the upper primary wall to define a storage compartment for unused feminine hygiene products;
the upper body being selectively moveable relative to the lower body between raised and lowered positions, at least a portion of the upper body being moved away from the lower body as the upper body moves from the lowered position toward the raised position to facilitate access to the disposal opening.

2. The waste disposal device recited in claim 1, wherein the door and the lower body are configured to form a fluid tight seal therebetween when the door is in the closed position.

3. The waste disposal device recited in claim 2, wherein the upper body and lower body are configured to form a fluid tight seal therebetween when the upper body is in the lowered position.

4. The waste disposal device recited in claim 1, further comprising a foot pedal coupled to the upper body, the foot pedal being configured to selectively move the upper body between the raised and lowered positions.

5. The waste disposal device recited in claim 1, wherein the foot pedal is pivotal relative to the lower body between a rest position and an actuated position, the upper body moving from the lowered position toward the raised position as the foot pedal moves from the rest position toward the actuated position.

6. The waste disposal device recited in claim 1, further comprising a lid coupled to the upper body, the lid being pivotable relative to the upper body between open and closed positions, the lid substantially covering the storage compartment when the lid is in the closed position.

7. The waste disposal device recited in claim 1, wherein the tubular lower wall defines a cylindrical shape.

8. The waste disposal device recited in claim 1, wherein the tubular lower wall includes a door portion and a stationary portion, the door portion being pivotable relative to the stationary portion to access the lower interior compartment.

9. The waste disposal device recited in claim 1, further comprising a restraining member engaged with the upper body and configured to be engageable with products stored in the storage compartment to mitigate movement of the products relative to the upper body as the upper body moves between the raised and lowered positions.

10. The waste disposal device recited in claim 1, further comprising a retaining member selectively engageable with the lower body for engaging a disposal bag with the lower body.

11. The waste disposal device recited in claim 1, wherein the door is biased toward the closed position.

12. The waste disposal device recited in claim 1, wherein the upper body is biased toward the lowered position.

13. A waste disposal device comprising:
a first body defining an inner waste compartment, the first body having a disposal opening formed therein in communication with the inner waste compartment;
a door pivotally coupled to the first body and pivotable between open and closed positions, the door being moved from the closed position to the open position to facilitate passage of waste products through the disposal opening and into the inner waste compartment; and
a second body pivotally coupled to the first body and defining a storage compartment, the second body being pivotable between raised and lowered positions, wherein a portion of the second body moves away from the first body as the second body moves from the lowered position to the raised position.

14. The waste disposal device recited in claim 13, wherein the door and the first body are configured to form a fluid tight seal therebetween when the door is in the closed position.

15. The waste disposal device recited in claim 14, wherein the second body and the first body are configured to form a fluid tight seal therebetween when the second body is in the lowered position.

16. The waste disposal device recited in claim 13, further comprising a foot pedal operatively coupled to the second body, the foot pedal being configured to selectively move the second body between the raised and lowered positions.

17. The waste disposal device recited in claim 13, further comprising a lid coupled to the second body, the lid being pivotable relative to the second body between open and closed positions, the lid substantially covering the storage compartment when the lid is in the closed position.

18. The waste disposal device recited in claim 13, wherein the first body includes a door portion and a stationary portion, the door portion being pivotable relative to the stationary portion to access the inner waste compartment.

19. The waste disposal device recited in claim 13, further comprising a retaining member selectively engageable with the first body for engaging a disposal bag with the first body.

20. The waste disposal device recited in claim 13, wherein the door is biased toward the closed position.
